(19) United States
(12) Patent Application Publication
(10) Pub. No.: US 2019/0380597 A1
(43) Pub. Date: Dec. 19, 2019

(54) DEVICE FOR MONITORING ACTIVITIES OF DAILY LIVING AND PHYSIOLOGICAL PARAMETERS TO DETERMINE A CONDITION AND DIAGNOSIS OF THE HUMAN BRAIN AND BODY

(71) Applicant: Newton Howard, Providence, RI (US)

(72) Inventor: Newton Howard, Providence, RI (US)

(21) Appl. No.: 16/441,527

(22) Filed: Jun. 14, 2019

Related U.S. Application Data
(60) Provisional application No. 62/685,647, filed on Jun. 15, 2018.
(60) Provisional application No. 62/686,203, filed on Jun. 18, 2018.

(51) Int. Cl. A61B 5/0205; A61B 5/01; A61B 5/0402; A61B 5/0476; A61B 5/00; A61B 5/11; A61B 5/1455; A61B 5/0285; G06F 3/16; H04R 1/10

(52) U.S. Cl. CPC A61B 5/02055; A61B 5/02125; A61B 5/0402; A61B 5/0476; A61B 5/0077; A61B 5/1116; A61B 5/14551; A61B 5/721; A61B 5/0285; A61B 5/6803; A61B 5/1118; G06F 3/165; G06F 3/167; H04R 1/1016; H04R 1/1041; H04R 1/1083; A61B 5/01

(57) ABSTRACT

Embodiments of the present systems and method may provide devices that can be conveniently worn continuously, yet monitor a wide range of physical and physiological parameters. For example, a system for monitoring human body activity may comprise a device mounted in an ear of a human, the device comprising a first portion adapted to be inserted in an ear canal of the human and a second portion adapted to protrude from the ear of the human, the first portion comprising a plurality of protrusions comprising at least one sensor, each sensor adapted to monitor a physical or physiological parameter of the human and output a signal, a data collection device adapted to receive the of signals and to process the signals to form digital data representing the monitored physical or physiological parameters, and a data processing device adapted to process digital data representing the monitored physical or physiological parameters.

### CROSS-REFERENCE TO RELATED APPLICATIONS

This application claims the benefit of U.S. Provisional Application No. 62/685,647, filed Jun. 15, 2018, and U.S. Provisional Application No. 62/686,203, filed Jun. 18, 2018, the contents of which are incorporated herein in their entirety.

### BACKGROUND

The present invention relates to a non-permanent integrated solution for unobtrusive monitoring of activities of daily living using a platform for biometrics.

Fitness tracking devices, such as tracking wristbands, watches, etc., have become popular for measuring and tracking certain activities of daily living, in particular, exercise or physical training activities. Such devices may measure certain physical or physiological parameters of the human body as they relate to exercise or training activities. These devices have the advantage of being capable of being conveniently worn 24/7, or at least for long periods, and thus provide long-term monitoring of the parameters. However, such devices only measure a few parameters. By contrast, medical monitoring devices are capable of monitoring many more parameters. However, such medical devices are generally too large and unwieldy to be used continuously during daily activities.

Accordingly, a need arises for devices that can be conveniently worn continuously, yet monitor a wide range of physical and physiological parameters.

### SUMMARY

Embodiments of the present systems and method may provide devices that can be conveniently worn continuously, yet monitor a wide range of physical and physiological parameters. For example, embodiments may provide a non-permanent integrated solution for unobtrusive monitoring of activities of daily living using a platform for biometrics. In an embodiment, a hearing aid headset for hearing—impaired patients may be provided. In an embodiment, a wireless audio streaming device and hands-free headset may be provided. In embodiments, in conjunction with, for example, a smartphone, embodiments may perform neural activity monitoring, such as electroencephalography (EEG), electrocardiography (ECG), measuring core body temperature, monitoring breathing, tracking activity, measuring blood oxygen saturation measurement (SpO2), monitoring blood pressure, etc.

For example, in an embodiment, a system for monitoring human body activity may comprise a device adapted to be mounted in an ear of a human, the device comprising a plurality of sensors, each sensor adapted to monitor a physical or physiological parameter of the human and output a signal representing the monitored physical or physiological parameter, a data collection device adapted to receive the plurality of signals from the plurality of sensors and to process the signals to form digital data representing the monitored physical or physiological parameters, and a data processing device adapted to process digital data representing the monitored physical or physiological parameters to determine a condition or activity of the human body.

In embodiments, the device adapted to be mounted in an ear of a human may further comprise a first portion adapted to be inserted in an ear canal of the human and a second portion adapted to protrude from the ear of the human, and the first portion comprises a plurality of protrusions, wherein at least some of the plurality of protrusions comprise at least one sensor. The sensors may comprise at least a plurality of sensors selected from a group comprising: audio sensors, video sensors, EEG sensors, ECG sensors, heart rate sensors, breathing rate sensors, blood pressure sensors, body temperature sensors, head movement sensors, body posture sensors, and blood oxygenation levels sensors. Each protrusion may comprise an electrically conductive rubber portion and an electrically isolated shell, wherein the electrically conductive rubber portion is adapted to be a dry electrode and to sense signals to be used for at least one of electroencephalography and electrocardiography. Each protrusion may further comprise microelectromechanical systems transducer comprising a mechanical transducer adapted to output an electrical signal representing a mechanical signal and an electrode adapted to output electrical signals received from a skin surface of the human body. The electrode may be a flexible electrode and the microelectromechanical systems transducer is further adapted to output an electrical signal representative of physical movement of the flexible electrode. The data processing device may be further adapted to determine artefacts of the physical movement that may be present in the electrical signal output from the flexible electrode, and to subtract the artefacts from the electrical signal output from the flexible electrode, to form a cleaner signal. The system may be further adapted to perform at least some of blood pressure measurement using Pulse Transit Time (PTT) and/or Pulse Wave Velocity (PWV), tympanic membrane infrared temperature measurement, accelerometer measuring of heart rate (HR), breathing rate (BR) and activity tracking, Photoplethysmography (PPG) optical measurement of blood volume changes, hearing aid functions, and music streaming capabilities with noise cancellation. The second portion may comprise a battery.

In an embodiment, a computer-implemented method for monitoring human body activity may comprise receiving from each of a plurality of sensors a signal representing a monitored physical or physiological parameter, wherein each sensor is adapted to monitor a physical or physiological parameter of the human and output a signal representing the monitored physical or physiological parameter, processing the received signals to form digital data representing the monitored physical or physiological parameters, and processing digital data representing the monitored physical or physiological parameters to determine a condition or activity of the human body.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 is an exemplary diagram of a device according to embodiments of the present systems and methods.

FIG. 2 is an exemplary diagram of a device according to embodiments of the present systems and methods.

FIG. 3 is an exemplary illustration of a Lithium Polymer (LiPo) battery according to embodiments of the present systems and methods.

FIG. 4 is an exemplary illustration of dimensions of an ear canal at isthmus.

FIG. 5 is an exemplary illustration of energy density of battery chemistries.

FIG. 6 is an exemplary diagram of a device according to embodiments of the present systems and methods.

FIG. 7 is an exemplary schematic for a common mode voltage buffer at the first amplifier stage according to embodiments of the present systems and methods.

FIG. 8 is an exemplary schematic of a front end circuit according to embodiments of the present systems and methods.

FIG. 9 is an exemplary diagram of a MEMS transducer device according to embodiments of the present systems and methods.

FIG. 10 is an exemplary diagram of a protrusion according to embodiments of the present systems and methods.

FIG. 11 is an exemplary illustration of ECG cycle.

FIG. 12 is an exemplary illustration of PTT (Pulse Transit Time) and PWV (Pulse Wave Velocity).

FIG. 13 is an exemplary illustration of accelerometer signals according to embodiments of the present systems and methods.

FIG. 14 is an exemplary illustration of magnetic field components.

FIG. 15 is an exemplary illustration of correlation between Photoplethysmography (PPG) and ECG.

FIG. 16 is an exemplary illustration of photon scattering in human tissue.

FIG. 17 is an exemplary illustration of statistical trajectory of photons in human tissue.

FIG. 18 is an exemplary diagram of a PPG measurement site according to embodiments of the present systems and methods.

FIG. 19 is an exemplary diagram of MEMS microphone functionality according to embodiments of the present systems and methods.

FIG. 20 is an exemplary block diagram of an analog MEMS microphone according to embodiments of the present systems and methods.

FIG. 21 is an exemplary block diagram of a digital MEMS microphone with PDM output according to embodiments of the present systems and methods.

FIG. 22 is an exemplary block diagram of a digital MEMS microphone with I2S output according to embodiments of the present systems and methods.

FIG. 23 is an exemplary block diagram of a double system MEMS active filtering microphone according to embodiments of the present systems and methods.

FIG. 24 is an exemplary illustration of a typical hearing aid speaker according to embodiments of the present systems and methods.

FIG. 25 is an exemplary illustration of a typical hearing aid speaker according to embodiments of the present systems and methods.

FIG. 26 is an exemplary illustration of arrangement of the probes on a device according to embodiments of the present systems and methods.

FIG. 27 is an exemplary illustration of arrangement of the probes on a device according to embodiments of the present systems and methods.

FIG. 28 is an exemplary illustration of a high-level mechanical drawing of an embodiment.

FIG. 29 is an exemplary illustration of a device according to embodiments of the present systems and methods.

FIG. 30 is an exemplary block diagram of a device according to embodiments of the present systems and methods.

FIG. 31 is an exemplary block diagram of a computer system in which processes involved in the embodiments described herein may be implemented.

### DETAILED DESCRIPTION

Embodiments of the present systems and method may provide a non-permanent integrated solution for unobtrusive monitoring of activities of daily living using a platform for biometrics. In an embodiment, a hearing aid headset for hearing—impaired patients may be provided. In an embodiment, a wireless audio streaming device and hands-free headset may be provided. In embodiments, in conjunction with, for example, a smartphone, embodiments may perform neural activity monitoring, such as electroencephalography (EEG), electrocardiography (ECG), measuring core body temperature, monitoring breathing, tracking activity, measuring blood oxygen saturation measurement (SpO2), monitoring blood pressure, etc.

Embodiments may include features such as rechargeable and replaceable Li—Po battery, dry electrodes made of conductive rubber for adherence and comfort, to sense signals for, for example, ECG and EEG monitoring. Further, embodiments may perform blood pressure measurement using Pulse Transit Time (PTT) and/or Pulse Wave Velocity (PWV), tympanic membrane infrared temperature measurement, accelerometer measuring of heart rate (HR), breathing rate (BR) and activity tracking, Photoplethysmography (PPG) optical measurement of blood volume changes, provision of hearing aid functions and/or music streaming capabilities with noise cancellation, etc.

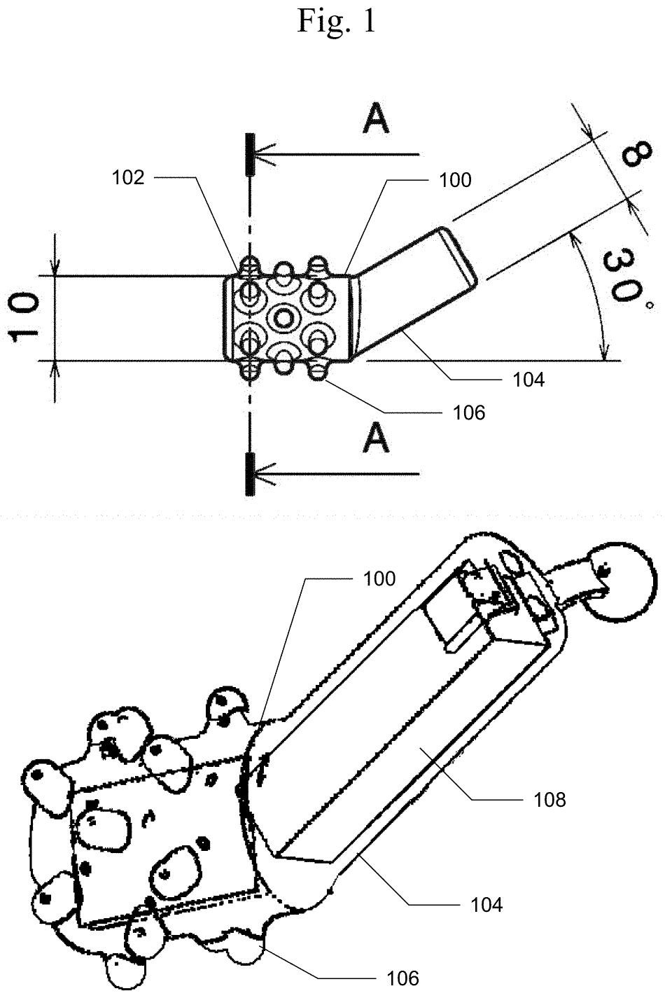

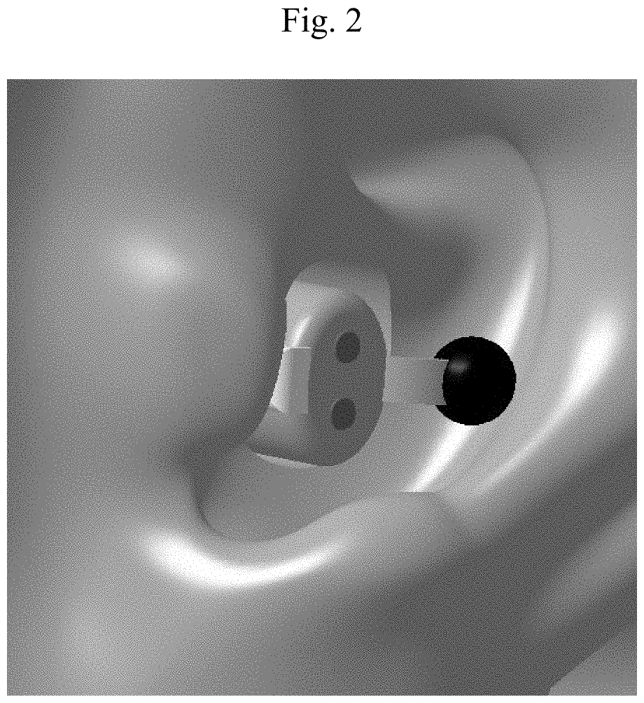

An exemplary embodiment of a device 100 is shown in FIG. 1. In this example, device 100 may include an inserted portion 102 and a protruding portion 104. Inserted portion 102 may be inserted in an ear canal during use, while protruding portion 104 may protrude from the ear during use, as shown in FIG. 2. Inserted portion 102 may include a plurality of protrusions 106, which may provide retention of device 100 within the ear canal. In this example, a battery or power cell 108 may be disposed within protruding portion 104.

Embodiments may use power sources such as disposable primary cells, or rechargeable batteries. For example, as EEG and ECG readings are relatively big power consumers, embodiments may use a rechargeable battery.

Embodiments may use different types of rechargeable batteries. For example, embodiments may use Lithium Titanate (Li₄Ti₅O₁₂) batteries. The main advantage of Lithium Titanate is the low working voltages, which means it can be directly connected to the circuitry of the device without voltage regulators, thus improving efficiency and reducing overall dimensions. Other advantages may include that, out of all the available chemistries, Lithium Titanate has the longest life span and can be easily found in off the shelf coin cells.

It appears that the maximum capacity which can be fitted using an available coin cell Lithium Titanate battery is 2.5 mAh. For comparison, rechargeable earbuds have a 25 mAh battery.

Embodiments may use a higher density battery, such as LiNiCoAlO₂. This chemistry has the disadvantage of working at higher voltages, thus needing a voltage regulator. They can be easily found in coin cells, but have the same problems as the Lithium Titanate battery, since these batteries cannot ensure significant energy in the available volume.

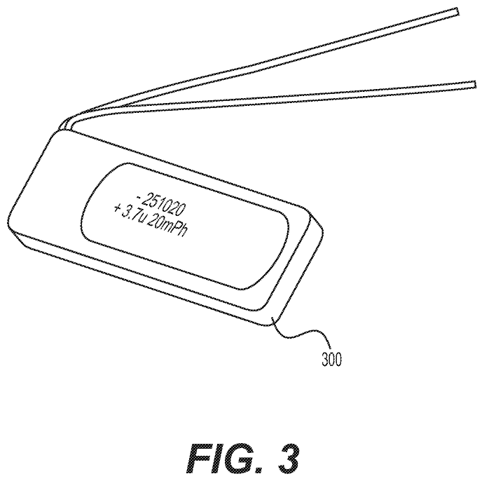

Embodiments may use a prismatic Lithium Polymer (LiPo) battery 300, an example of which is shown in FIG. 3, which may conform to shapes more fitting to the available space, and are available in various sizes and on customer specifications. LiPo batteries have high energy density and are used in virtually all Bluetooth headsets. FIG. 3 depicts a typical LiPo pouch battery 300 used in Bluetooth headsets.

In embodiments, the battery size may be chosen based on the exact purpose and dimensions of the device. For example, in some cases a battery of the specified size would not physically fit, given the location in which the device is to be used.

In embodiments, the device may extend past the opening of the ear canal, creating space for a much larger battery. For example, in embodiments, a pouch type battery may be used, which will make the device pass through the isthmus up to the surface of the ear, as seen in FIG. 2. In order to increase the volume available for hardware components, it appears the isthmus of the inner ear is the biggest restriction. Therefore, embodiments may include a battery that fits inside the isthmus, extends to the outer surface of the ear, and is flush with the tragus.

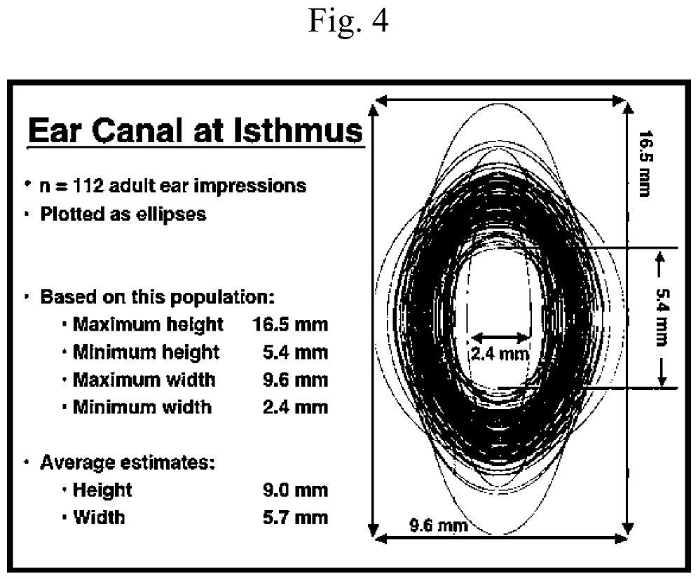

For example, a study done on 112 adults revealed the dimensions presented in FIG. 4. As the isthmus is elastic, given the average dimensions shown in FIG. 4, embodiments may utilize an 8×6 mm oval shape that allows a parallelepiped pouch battery to fit in the oval shell. In order to obtain a maximum surface area which fits in the oval shape, the area of the rectangle may be expressed as a function which solves the oval equation:

$$\frac{{x}^{2}}{16} + \frac{{y}^{2}}{9} = 1.$$

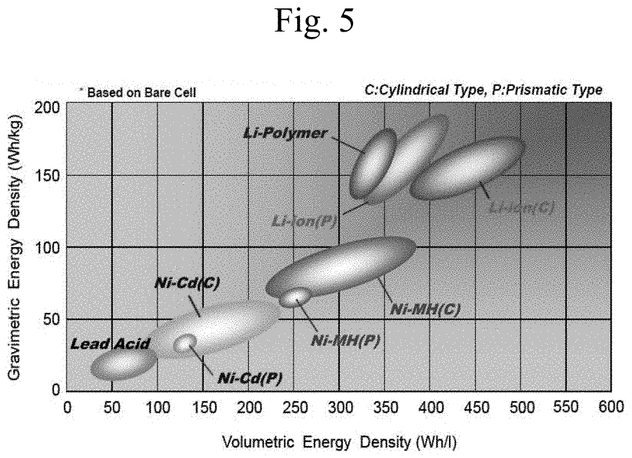

Then, by differentiation, a value for x=5.65 and y=4.24 mm may be obtained. After 3D modelling, a length of 18 mm may be found. Accordingly, embodiments may use a battery having dimensions of about 5.65×4.24×18 mm battery, which may provide a usable volume of about 431 mm³. Given that the lithium polymer chemistry provides between 330-430 Wh/l, as shown in FIG. 5, embodiments may use a 400 Wh/l battery. Accordingly, embodiments may use batteries of approximately 175 mWh, or, at a nominal voltage of 3.7V, 47 mAh. Further, embodiments may use more exotic chemistries to obtain as much as 3 times more energy in the same volume.

Embodiments may use Bluetooth 5 rather than the older 4.2 protocol, which almost doubles the battery life. Therefore, embodiments may provide a battery life of approximately 10 h when playing music continuously. In order to allow for battery replacement, in embodiments, the shell of the device may be split longitudinally, thus enabling battery access through the removable cover.

Electric Signal Acquisition. Electrodes. Dry versus Wet Electrodes. Embodiments may use wet electrodes, while other embodiments may use dry electrodes. The advantage of using wet electrodes is the contact resistance between the skin and the electrode is far lower, as it can be seen in Table 1. For example, a method to determine the equivalent resistance of the wet/dry electrodes may provide more relevant information about the resistance difference between electrode types and not about the absolute value of their resistance. For example, two wet electrodes may be placed on the forearm 6 inches apart. Subsequently, one wet electrode may be replaced with a dry electrode in order to quantify the resistance variation. The measurements may be made in AC at frequencies between 5 Hz and 100 Hz. Table 1 below shows the resistance of the electrodes at different test frequencies.

TABLE 1

| | Frequency (Hz) | | | | | | | | | | | | |
|---|---|---|---|---|---|---|---|---|---|---|---|---|---|
| | 5 | 7.5 | 10 | 15 | 25 | 35 | 45 | 55 | 65 | 75 | 85 | 95 | 100 |
| Wet Resistance (MOhm) | .24 | .16 | .13 | .12 | .09 | .07 | .05 | .05 | .04 | .04 | .04 | .04 | .03 |
| Dry Resistance (MOhm) | .52 | .45 | .43 | .41 | .39 | .39 | .39 | .37 | .37 | .37 | .37 | .36 | .36 |

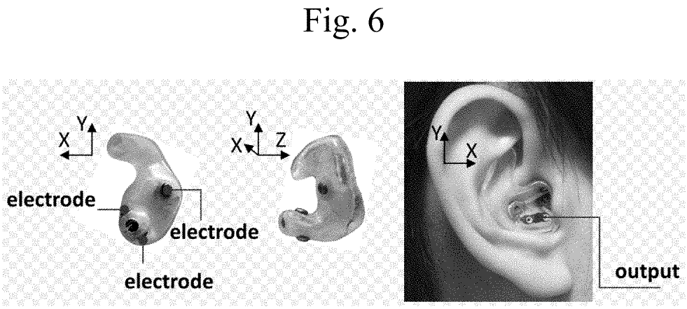

However, from a design perspective, the dry electrodes may be more comfortable to wear as well as be easier to maintain by the user. The wet contact requires a special gel that feels uncomfortable for many users, requires more cleaning and blocks the flow of oxygen to the tympanic membrane. For example, FIG. 6 depicts an exemplary embodiment for a device that uses dry electrodes.

In addition, the sebaceous fluid, dead skin, and contact pressure variation may cause temporary changes in DC offset. This kind of noise is difficult and almost impossible to reject because its frequencies fall into the bandwidth of interest. Accordingly, the first differential amplifier may provide a circuit that injects the common voltage into an electrode.

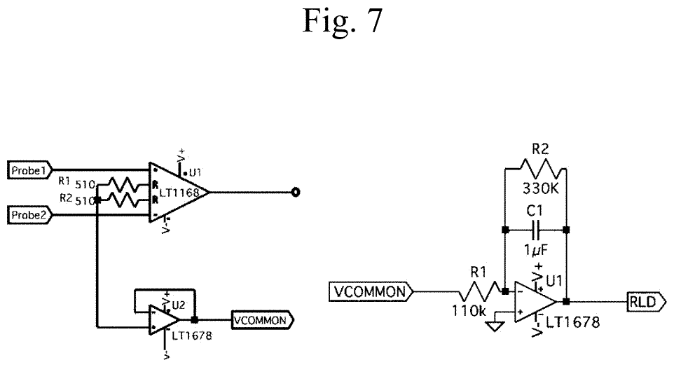

An exemplary schematic for a common mode voltage buffer at the first amplifier stage is shown in FIG. 7 below. The RLD terminal means Right Leg Drive. This term comes from ECG technology where the right leg is driven to a known potential to avoid interfering with the heart operation. In this case the RLD sets a common mode voltage to improve the common mode voltage rejection of the acquisition system. An important noise source in such systems is the 50/60 Hz perturbation from domestic power lines. As a consequence, high quality notch filters may be introduced in the signal path.

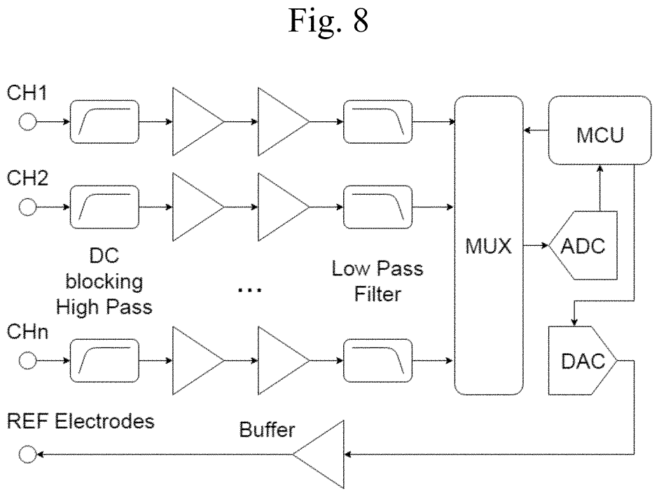

In embodiments, the front end circuit may be implemented as in the following structure illustrated in the example shown in FIG. 8, or may be integrated in a system containing the Bluetooth communication transceiver, ADC, and MCU. A starting point may be the first differential amplifier stage and the common mode circuit with DC blocking filters as an external block. In embodiments, the rest of the filters may be implemented in software for space saving.

Size of Dry Electrodes. In order to maintain a low contact resistance, the electrodes may ensure firm contact with the skin. The size of the dry electrodes is bigger than that of the wet electrodes, but they usually have elements like spikes that use a very low contact area with the skin. Care must be taken when using sharp spikes, as this can create pain and discomfort.

When choosing electrodes for biometric systems, mechanical aspects such as dimensions and ergonomics may be considered. Some of the advantages and disadvantages of the dry electrodes with respect to their dimensions are presented in Table 2 below, which shows a comparison between different dry electrode sizes.

TABLE 2

| Scale | Advantage | Disadvantage |
|---|---|---|
| Nano | Similar impedance with wet electrodes | Invasive |
| | No risk of infection | Not good for hairy |
| | Less motion artifacts | sites |
| Micro | Similar impedance with wet electrodes | Invasive |
| | Less motion artifacts than millimetric | Risk of infection |
| | scale | Fragile |
| | | Not good for hairy |
| | | sites |
| Mili | Non invasive | Artefacts due to |
| | No risk of infection | motion |
| | Good for hairy sites | Higher impedance |

Table 3 below shows a list of commercial devices that use dry electrodes and their main characteristics and properties.

TABLE 3

| Name | Purpose | Description | Vendor |
|---|---|---|---|
| Sahara | BCI | Dry, active electrode system that works | g.tec medical |
| | | for all frontal, central, occipital, and | engineering |
| | | parietal sites. Electrode composed of 8 | GmbH |
| | | pins made of gold alloy. Bandwidth: 0.1- | |
| | | 40 Hz. When used with Nautilus: | |
| | | Sampling rate: 500 Hz. Up to 32 | |
| | | channels. 3-axis acceleration sensor. | |
| Insight | BCI | A 5 channel (plus 2 references) wireless | Emotiv |
| | | headset to track and monitor brain activity | |
| | | and stream to mobile devices. Although | |
| | | the advertisement states it is a dry EEG | |
| | | system, the technical specifications state | |
| | | the sensors are made of semi-dry polymer. | |
| | | Bandwidth: 1-43 Hz, Sampling rate: 128 | |
| | | Hz, Wireless interface: Bluetooth 4.0 LE. | |
| DSI 10/20 | BCI | Ultra-high impedance sensors (47 GQ). | Quasar |
| | | Up to 23 electrodes at a sampling rate of | |
| | | 960 Hz and a maximum bandwidth of 120 | |
| | | Hz. Suitable for locations with hair | |
| Brain Band XL | BCI | Dual sensor EEG unit (one active with | MindPlay |
| | | adjustable positions). Bluetooth | |
| | | Connectivity. Sampling rate 512 Hz and | |
| | | bandwidth up to 50 Hz. Automatic with | |
| | | processing of attention, meditation, and | |
| | | eye blink detection. Based in TGAM | |
| | | sensor by Neurosky. Not suitable for | |
| | | locations with hair. | |
| XWave Headset | BCI | Neuro Sky eSense Dry Sensor. Not | PLX Devices |
| | | suitable for locations with hair. | |
| Enobio | BCI | UP to 20 channels at a sampling rate of | Starlab |
| | | 500 Hz. Wireless operation with | |
| | | Bluetooth and 50 nV of quantification step | |
| Mindflex | Electronic | Based on attention and meditation to | Mattel |
| | Game | control the vertical position of a plastic | |
| | | ball by activation of a fan underneath. It | |
| | | uses TGAM by Neurosky | |
| EEG Headset | Health | 8-channel EEG monitoring chipset. Each | Imec |
| | monitor | EEG channel consists of two active | |
| | | electrodes and a low-power analog signal | |
| | | processor with high input impedance (1.4 | |
| | | GΩ at 10 Hz) | |
| ThinkGear AM | Gaming | Non-contact dry sensor. Sampling rate | Neurosky |
| EEG | | 512 bits. Bandwidth 3-100 Hz. Operates | |
| | | at a minimum of 2.97 V. It works with | |
| | | Ag/AgCl, Stainless Steel, Gold, or/and | |
| | | Silver electrodes. It outputs attention, | |
| | | meditation, and eye blinks. Not suitable | |
| | | for locations with hair. | |
| Dry Pad | BCI | Reusable Ag/AgCl EEG pad electrode | Cognionics |
| | | suitable for locations without hair. | |
| | | Electrode impedance 10-100 KΩ. The | |
| | | active version only needs a supply battery | |
| | | of 1.8 V. Small size (versions with 2-5 | |
| | | cm diameter circa.). | |
| Flexible Dry | BCI | Flexible and reusable (up to 30 sessions) | Cognionics |
| EEG | | Ag coated elastomer. Suitable for | |
| | | locations with hair. Electrode impedance | |
| | | 100-2000 KΩ. | |
| Muse | Stress | Seven EEG electrodes built into a | Interaxon |
| | monitoring | headband. Sampling rate 600 Hz. | |

Electrically conductive silicone rubber, such as that manufactured by SHIN ETSU®, may be specified with volume resistivities between 0.009 Ωm and 0.05 Ωm.

Movement artefacts. Electrode technologies established in many clinical settings are typically developed to obtain low electrical impedance between body and instrumentation equipment. In practice, one of the biggest challenges associated with physiological recordings are the motion artefacts induced by relative movements between the electrode and the skin, which affect the electrochemical electrode-skin interface, thus causing interferences. Despite a significant effort to develop mechanically stable electrode-skin interfaces, current electrodes are still prone to motion artefacts as well as skin stretch.

In order to satisfy the “wearable” requirement, physiological recordings need to be performed without the conductive gel. Even if movements of a subject are constrained in a controlled environment, modern electrodes frequently provide suboptimal signal quality. This is particularly detrimental with the elderly and those suffering from neurodegenerative diseases (e.g. Parkinson's disease).

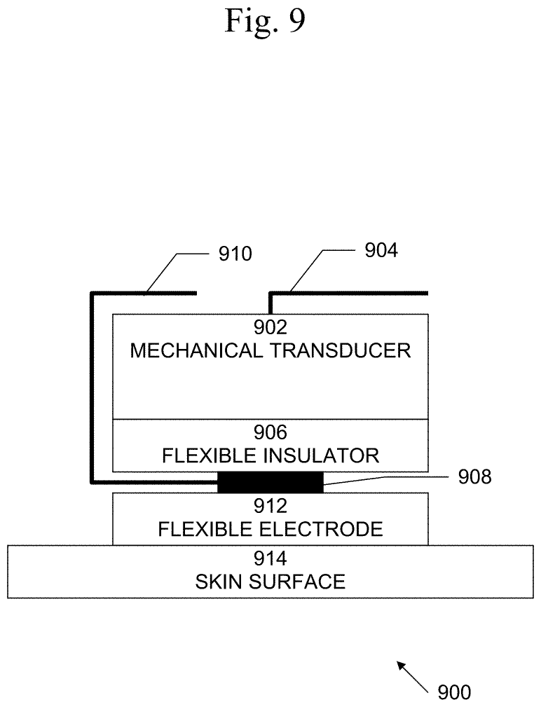

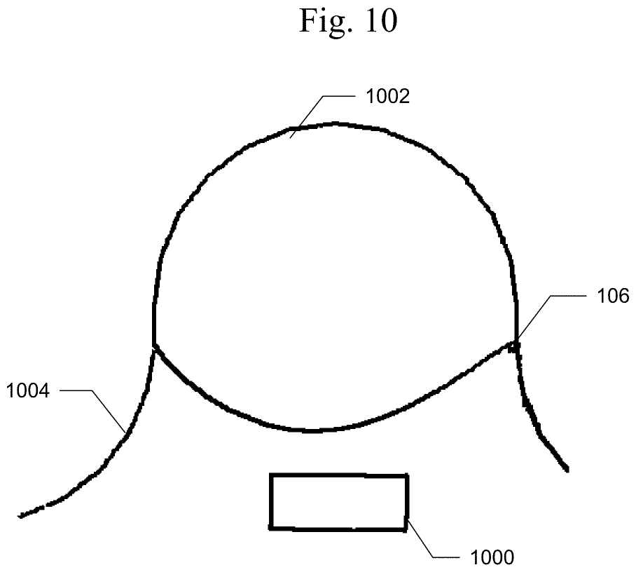

While electrodes suffer from skin-contact movement, these artefacts may be rejected using input from correlated sensors, such as Microelectromechanical systems (MEMS) transducers, such as the example shown in FIGS. 9 and 10. In this example, multimodal MEMS sensor 900 may measure electrical and mechanical responses from the same location. MEMS sensor 900 may include a mechanical transducer 902, which may output an electrical signal 904 representing a mechanical signal. Flexible insulator 906 may separate mechanical transducer 902 from conductive copper wire 908, which may communicate electrical signal 910 from flexible electrode 912, which may be in electrical contact with conductive copper wire 908. Flexible electrode 912 may be in electrical contact with, and may receive electrical signals from, skin surface 914. As mechanical transducer 902 is in physical contact with flexible electrode 912, mechanical transducer 902 may output an electrical signal 904 representative of physical movement of flexible electrode 912. Estimates of the artefacts of the physical movement that may be present in the signal 910 output from flexible electrode 912 may be computed based on signal 904 using signal processing and subtracted from signal 910, which may be a corrupted ECG signal, to obtain a relatively clean, or at least cleaner, signal.

An example of placement of a MEMS sensor 1000 in a device 100, such as that shown in FIG. 1, is shown in FIG. 10. Protrusion 106 may include an electrically conductive rubber portion 1002 and an electrically isolated shell 1004. In this example, a protrusion 106 is shown, with MEMS sensor 1000 located near the base of protrusion 106. However, MEMS sensor 1000 may be located at any position on or near protrusion 106.

ECG. Electrocardiography is the process of recording the electrical activity of the heart over a period of time using electrodes placed on the skin. These electrodes detect the tiny electrical changes on the skin that arise from the heart muscle's electrophysiologic pattern of depolarizing and repolarizing during each heartbeat. It is commonly performed to detect any cardiac problems.

In embodiments, ECG may provide the capability for examination of heart conditions that are visible in multiple consecutive cardiac cycles. The conditions include, for example, myocardial infarction (reflected in an elevated ST segment), first-degree atrioventricular block (the PR interval is longer than 200 ms), atrial fibrillation (the P-wave disappears, found in 2% to 3% of the population in Europe and the USA), sinus tachycardia (elevated regular heart rate, P-wave can be close to the preceding T-wave) and atrial flutter (atria contract at up to 300 bpm, atrioventricular node contracts at 180 bpm, frequency of P-waves is much higher than the frequency of QRS-complexes). Embodiments may provide a framework for 24/7 continuous and unobtrusive cardiac monitoring and recording. Depending on the available power budget and the indications from a medical professional regarding ECG analysis, the monitoring time may be reduced down to few measurements per day.

Embodiments may provide insight into the activity of the autonomic nervous system and its components, the sympathetic and parasympathetic nervous systems, and may act as an early-warning and tele-monitoring system for certain cardiovascular diseases.

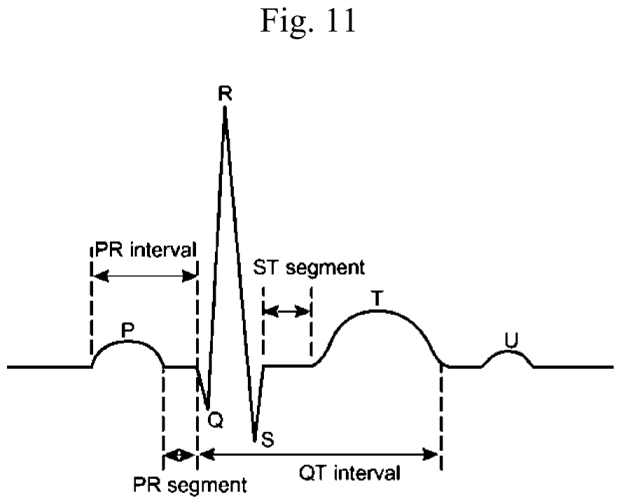

An example of an ECG cycle is shown in FIG. 11, and a description of its features are given in Table 4 below.

TABLE 4

| Section | Description |
|---|---|
| P-wave | Atrial depolarization or contraction; Duration: 60-120 ms |
| PR-interval | Time taken for the impulse to spread into the atria; |
| | Preceding ventricular contraction; Duration 120-200 ms |
| QRS-complex | Duration: less than 30 ms |
| QRS-interval | Depolarization of both ventricles (systole); Duration: |
| | less than 120 ms |
| ST-segment | Time between ventricular depolarization and |
| | repolarization (diastole); Duration: 120 ms |
| T-wave | Ventricular repolarization; Duration: 160 ms |
| QT-interval | Entire electrical depolarization and ventricular |
| | repolarization; Duration: 340-430 ms |
| U-wave | Repolarization of Purkinje fibers in the papillary muscle |
| | of the ventricular myocardium; Visible when heart rate is |
| | slow. |
| TP-segment | Used just as a reference point |

EEG. Electroencephalography is a noninvasive method for analyzing and recording the electrical activity of the brain. Usually the signal sampling is made by placing an electrode grid on the scalp. The electrical activity of the brain is caused by the fluctuations resulting from ionic current within the neurons. Based on a clinical study, the signal amplitude at the scalp electrodes fits in the 10-100 μV range for an adult. The frequency band required to measure such signals starts from 1 Hz up to 70 Hz. Within this frequency band the cerebral activity falls into different signal EEG frequency band categories as shown in Table 5 below.

TABLE 5

| Wave type | Signal band | Location | Trigger activity |
|---|---|---|---|
| Delta | 0.5 Hz-4 Hz | Frontal cortex | Slow wave sleep |
| Theta | 4 Hz-7 Hz | Hippocampus | When repress a response or action |
| Alpha | 7 Hz-15 Hz | Occipital lobe | Closing the eyes when relaxing |
| Beta | 15 Hz-31 Hz | Mostly frontal | Active thinking, focus |
| Gamma | Over 31 Hz | Somatosensory cortex | Hearing, sight, short term memory |
| Mu | 8 Hz-12 Hz | Sensorimotor cortex | Rest state motor neurons |

In order to sample such weak signals, special care has to be taken concerning signal integrity and electromagnetic compatibility of the circuitry. A high impedance acquisition channel is prone to parasitic couplings and induced noise.

In embodiments, the sampling system may have three main parts: Band pass filters for DC blocking and bandwidth limitation, Gain stages made out of 2 or 3 amplifiers, and an analog-to-digital converter (ADC).

In embodiments, an earset may include three dry electrodes for EEG recording. Two differential electrodes may be fitted into the ear canal. Another external reference electrode may be connected to concha cavum site of the ear.

Blood Pressure. The conventional method for blood pressure (BP) monitoring involves a manometer, a stethoscope and a cuff which temporarily cuts off the blood flow to the hand. This is an unsuitable method for continuous BP measurement.

Techniques for blood pressure measurement in a wearable device may depend on the location of the device on the human body. For example, measuring BP on the wrist requires continuous calibration due to the changing hydrostatic pressure relative to the heart. Placing the device on extremities makes the acquisition system more susceptible to noises coming from subject movement. When placing the system inside the ear, its position is more stable because the ear provides a natural anchoring point.

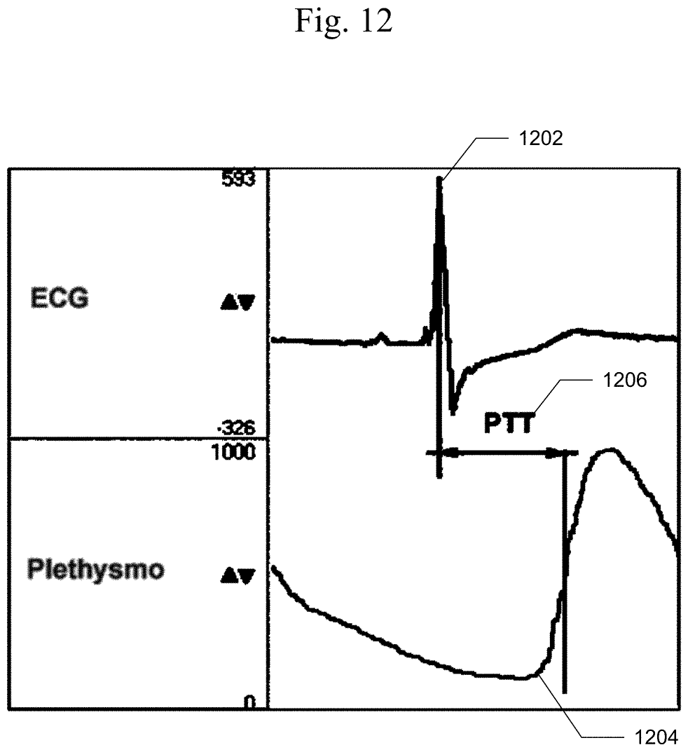

An exemplary comparison between the classical BP monitoring method with cuff and the a method based on calculating the blood pressure using PTT (Pulse Transit Time) and PWV (Pulse Wave Velocity) is shown in FIG. 12. In this example, one can see the time shift between the R wave spike 1202 in the ECG and the pulse wave arrival at the periphery (designated PTT 1206).

The cuff free method may be connected with the ECG and blood oximetry data. The pulse transit time is defined as the time shift between R spike 1202 on the ECG and the plethysmographic curve 1204 of an arterial tissue oximetry. Improved results may be obtained when sensing a relatively big artery such as in the hand. If the device is place inside the ear channel, the blood oxygen saturation may be measured with a reflective method. The PWS (pulse wave velocity) can be expressed with the equation below:

$$PWV ( cm \text{/} ms ) = \frac{BDC \times height ( cm )}{PTT ( ms )}$$

BDC represents the body correlation factor. For example, when detecting the peripheral pulse at the finger of an adult, this parameter has a value of 0.5. This parameter needs to be tuned, depending on the position of the pulse detection, height, and age of the patient. The relation between PWV and the BP may be approximated with the following formula:

BP_{PTT}=P1×PWV×e^{(P3×PWV)}+P2×PWV^{P4}−(BP_{PTT,cal}−BP_{cal})

BP_{PTT,cal} is an indirect blood pressure measurement method using PTT. BP_{cal} is the trusted reference blood pressure. The parameters P1 to P4 are parameters estimated by least square fitting of the data coming from the subjects.

Body Temperature. Since the hypothalamus at the brain's base regulates the core body temperature, this is the golden standard for temperature measurement. As the ear canal's eardrum blood vessels are shared with the hypothalamus, embodiments may include an infrared sensor to measure the tympanic membrane temperature.

Table 6 below presents examples of possible options for this component:

TABLE 6

| | | |
|---|---|---|
| Melexis | MLX90632 | 3 × 3 × 1 mm |
| Texas Instruments | TMP007 | 1.9 × 1.9 × |
| | | 0.625 mm |
| Texas Instruments | TMP006 | 1.5 × 1.5 mm |

Control, Power and Communications. As the device collects data from the sensor, this data may be recorded, processed, stored, and transmitted. Table 7 shows examples of commercially available Systems on Chip (SOC) which include communication and processing modules.

TABLE 7

| | | | | | | ESP32- |
|---|---|---|---|---|---|---|
| | QN908x | QN9022 | CC2564MODx | nRF52810 | IS1871 | PICO-D4 |
| Dimensions | 3.2 × 3. | 5. × 5. | 7. × 7. × | 2.48 × 2.46 | 4. × 4. × | 7. × 7. |
| mm | | | 1.4 | | 0.9 | |

Additionally to the SOC, embodiments may include a Digital Signal Processor (DSP), as the EEG requires high order filters and the DSP may further be helpful in sound processing. Table 8 shows examples of SOCs with DSP support.

TABLE 8

| | EFR32 | | CC2640R2F | | DA14586 |
|---|---|---|---|---|---|
| | Blue Gecko 32 | RSL10 | SimpleLink | CSR8670 | Dialog |
| | (Siliabs) | (ON Semi) | (T.I.) | Qualcomm | Semiconductor |
| Dimensions | 3.3 × 3.14 mm | 2.35 × 2.32 | 2.7 × 2.7 mm | 4.7 × 4.8 mm | 5 × 5 mm (QFN |
| (mm) | (WLCSP43) | (WLCSP- | (14GPIOs) | (WLCSP) | 40) |
| | BGA125 | 51) | DSBGA34 | | |
| | (7 × 7 mm) | | | | |
| DSP | yes, integrated | yes, | no | yes | no |
| | in MCU | LPDSP32 | | | |

Given this information, embodiments may include the ON Semiconductor RSL10 IC (Integrated Circuit). However, embodiments may include any of the indicated components, or any other components that may provide similar or equivalent functionality.

Accelerometer. An accelerometer may be provided in order to correlate heart rate (HR) and breathing rate (BR) with collected motion data. Also, information such as gait or median activity frequency may be obtained. Some signals, such as heart rate and breathing rate may be correlated with data taken from other sensors, such as the optical system used for pulse oximetry.

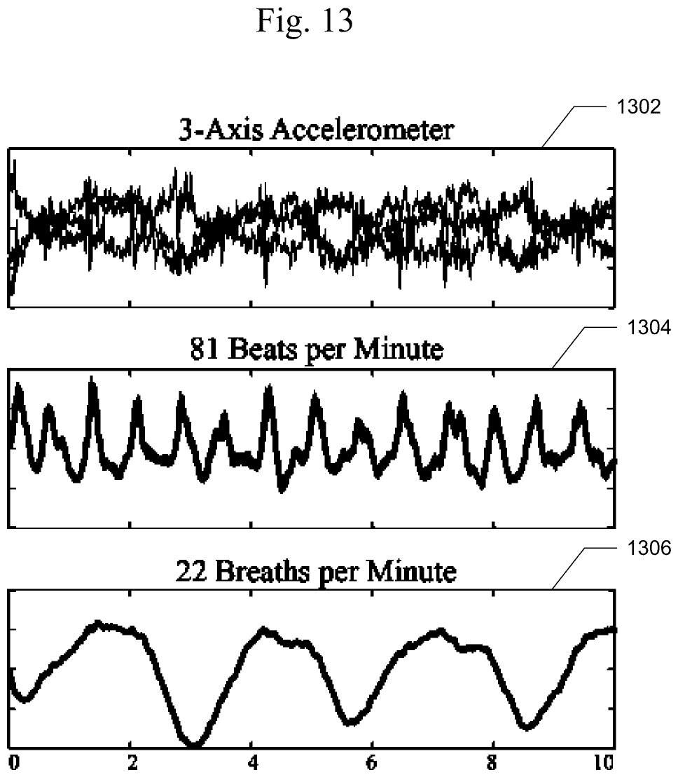

Using the onboard DSP, embodiments may filter the signals in order to separate the data of interest, using their known characteristics, such as frequency and amplitude, compared to a known baseline. An example of this approach is shown in FIG. 13, which shows measured 1302 and filtered 1304, 1306 accelerometer signals.

Examples of accelerometers are shown in Table 9 below:

TABLE 9

| Parameter | Unit | ADXL362 | BMA455 | KX112 | MC3571 | MMA8451Q | LIS2DS12 |
|---|---|---|---|---|---|---|---|
| Size | mm³ | 3 × 3.25.1.06 | 2 × 2 × 0.65 | 2 × 2 × 0.6 | 1.085 × 1.085 × | 3 × 3 × 1 | 2 × 2 × 0.86 |
| | | | | | 0.74 | | |
| Max. FS | g | ±8 | ±16 | ±8 | ±16 | ±8 | ±16 |
| 0-g Offset | mg | ±35 | ±50 | ±25 | ±80 | ±20 | ±30 |
| Offset T. Co. | mg/° C. | 0.5 | NA | ±0.2 | ±1 | ±0.15 | ±0.2 |
| Resolution | bits | 12 | 14 | 8, 16 | 8, 10, 14 | 8, 14 | 10, 12, 14 |
| Sensitivity/SF | mg/LSB | 1 | 0.244 | 0.061 | 0.244 | 0.244 | 0.061 |

Embodiments may include, for example, the MC3571, or other suitable accelerometer.

Wireless Power Transfer. There is an important opportunity for the earbuds to be used in the Neuron on Augmented Human system, as a temporary power station for the brain implant. In embodiments, the earbud may be used as a wireless charger for a brain implant, given the specific dimension constraints.

The equations below describe how the wireless charger transfers energy from transmitter to the receiver coil. The first equation expresses the magnetic flux density generated by the transmitting coil in a point P situated on the same central axis at distance x. The flux density is a function dependent on windings number, coil diameter and the current that flows through it.

$${B}_{x} = \frac{{μ}_{0} {NIr}^{2}}{2 {( {x}^{2} + {r}^{2} )}^{3 / 2}}$$

$${φ}_{m} = \int BdS$$

$$V ( t ) = - \frac{{Ndφ}_{m} ( t )}{dt}$$

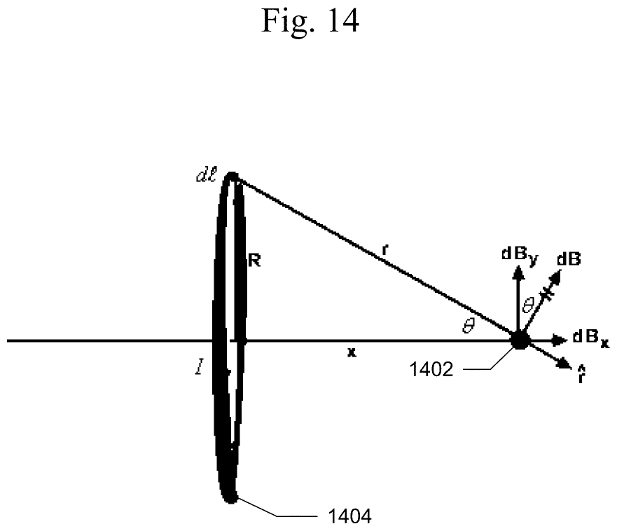

FIG. 14 illustrates the relation between the magnetic field components in a point 1402 situated at distance x from the transmitting coil 1404. Based on the last equation, the induced voltage into a receiver coil may be calculated as a function of the number of turns, the gap between coils, and the frequency. Considering a wireless charging system having two identical inductors with 25 turns, 6 mm diameter, with an air-gap of 6 mm, the voltage induced in the receiver coil reaches only 8 mV. The value is way too low for a feasible scenario. The transmitter coil was energized with 25 mA RMS current. As a result, wireless charging can't be implemented with the actual battery capacity of 40 mAh and the space inside the ear channel. Table 10 below shows receiver voltages at different system parameters (nOK=Not OK), such as different air-gaps, coil diameters and excitation current combinations:

TABLE 10

| Transmitter coil | | | | | | Receiver coil | | | | |
|---|---|---|---|---|---|---|---|---|---|---|
| Radius | | Current | gap | | freq | Radius | | | | |
| [mm) | Turns | [mA] | [mm] | Field [B] | [kHz] | [mm] | Turns | Area (m²) | Voltage [mV] | |
| 3 | 25 | 25 | 100 | 3.5E−09 | 150 | 3 | 25 | 2.8274E−05 | −0.002351364 | nOK |
| | | | | | | | | | cos(2 * pi * f * t) | |
| 3 | 25 | 25 | 50 | 2.8E−08 | 150 | 3 | 25 | 2.8274E−05 | −0.018735053 | nOK |
| | | | | | | | | | cos(2 * pi * f * t) | |
| 3 | 25 | 25 | 25 | 2.2E−07 | 150 | 3 | 25 | 2.8274E−05 | −0.14749321 | nOK |
| | | | | | | | | | cos(2 * pi * f * t) | |
| 3 | 25 | 25 | 12 | 1.9E−06 | 150 | 3 | 25 | 2.8274E−05 | −1.244138608 | nOK |
| | | | | | | | | | cos(2 * pi * f * t) | |
| 3 | 25 | 25 | 6 | 1.2E−05 | 150 | 3 | 25 | 2.8274E−05 | −7.799866018 | nOK |
| | | | | | | | | | cos(2 * pi * f * t) | |
| 4 | 25 | 800 | 4.4 | 0.00096 | 300 | 4 | 25 | 5.0265E−05 | −2265.022128 | OK |
| | | | | | | | | | cos(2 * pi * f * t) | |
| 50 | 40 | 25 | 83 | 1.7E−06 | 300 | 2 | 25 | 1.2566E−05 | −1.022449494 | nOK |
| | | | | | | | | | cos(2 * pi * f * t) | |

In order to induce at least 2.2 V at the receiver coil at a 4.4 mm air-gap, it is necessary to energize the transmitting coil with a current of at least 800 mA @ 300 kHz. The coil diameters shall be higher than 8 mm.

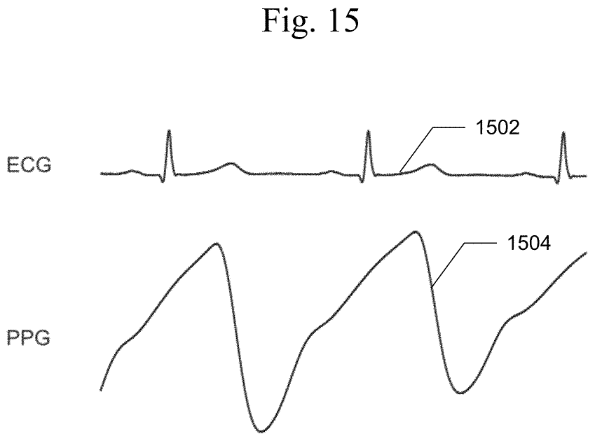

Photoplethysmography (PPG) is a simple optical method that can be used to detect changes in blood volume flowing through the microvascular tissue. Using this technique we can make non-invasively measurements at the skin surface. The PPG waveform is comprised of a pulsatory waveform, typically around 1 Hz, attributed to cardiac changes in the blood volume synchronized with each heartbeat, and is superimposed on a slowly varying baseline with various lower frequency components attributed to respiration, sympathetic nervous system activity and thermoregulation. With suitable amplification and filtering, be it electronic or digital, all these signals can be extracted for subsequent pulse wave analysis. FIG. 15 illustrates such a correlation between PPG and ECG, showing the pulsatile (AC) component of the PPG signal 1502 and corresponding electrocardiogram (ECG) 1504.

Light interaction with biological tissue may include scattering, absorption, reflection, transmission and fluorescence, and the key factors that can affect the amount of light received by the photodetector may include blood volume, blood vessel wall movement and the orientation of red blood cells.

Due to the fact that embodiments of the present device may be compact and comfortable, a reflexive measurement approach may be used, as this allows placement of optic source and detector on the same side of the skin surface. Two main factors need to be addressed in order to gather high quality data. One factor is that the tissue is highly forward scattering, which results in the signal quality of reflection mode being no better than that of the transmission mode The other factor is related to the method used to determine the distance between the light source and photodetector. Embodiments may be address this factor by using a multimodal sensor design, where data from the photodetector may be correlated with data from an electrical probe at the same site. Embodiments may integrate a MEMS pressure transducer at the base of the optical assembly.

As human tissue is a strongly scattering media, in which a photon may propagate along a random path 1602, as is shown in FIG. 16, most of the photons may be scattered repeatedly before escaping outside the tissue surface.

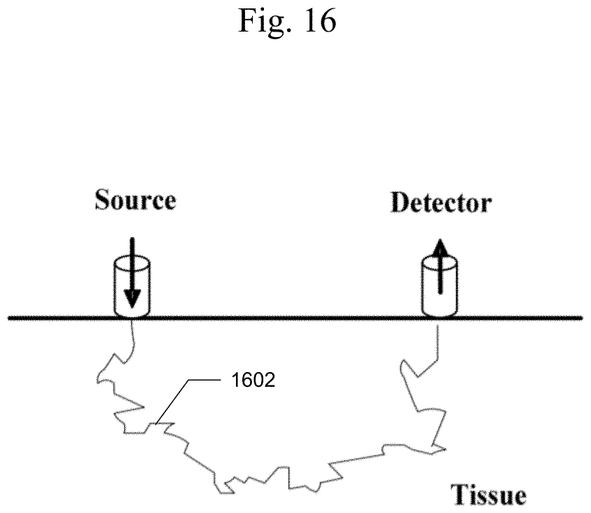

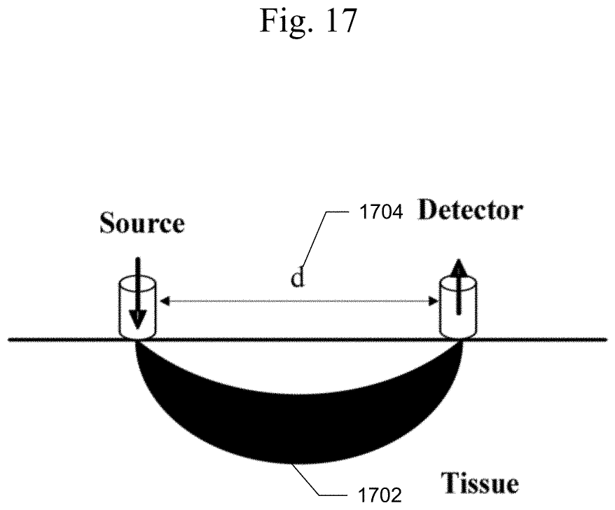

Although the paths of different photons propagating in the highly scattering human tissue may not be the same, the statistical trajectory of the photons between the emitter and detector may conforms to a banana-shape path area 1702, as shown in FIG. 17.

In order to effectively study the properties of the tissue layer of interest, there should be as many as possible photons that propagate through it. The detection depth varies with the source-detector separation 1704, which may be optimal when the corresponding penetration depth just reaches the bottom of the interested tissue layer.

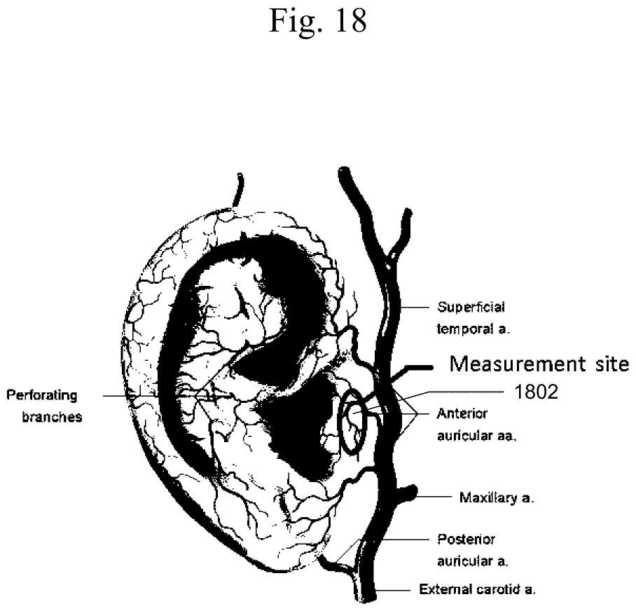

Embodiments may include an optical instrument composed of a photodetector surrounded by LEDs. For example, an optical assembly may press against the PPG measurement site, which may be the inner tragus 1802, as shown in FIG. 18.

Microphone. Many commercial hearing aids use at least two omnidirectional microphones in order to offer proper audible experience to user, with the scope of obtaining sound directionality. A Digital Hearing Aid processes the speech signal in the same manner as the human ear functions. Factors against using two microphones rather than one for obtaining directivity and better speech understanding may include additional costs and extra space need for extra calibration, while factors that favor using two microphones may include better speech understanding in noisy environments, as no signal processing technology can deliver such great improvement in directional signal processing as two microphones (name front/rear), improved signal to noise ratio, and directional filtering that is independent of the type of the noise

Reverberation is an effect to be taken into account when implementing a single microphone vs. dual omnidirectional microphone technology in hearing aids. It is known that cochlear implants are more affected by reverberation than conventional hearing aids.

Embodiments may include features for directional filtering, such as a Fixed Directional Pattern and/or an Adaptive Beamformer System. Signal processing methods typically used in hearing aids may include adaptive filtering, frequency domain shifting, feedback and echo cancellation, dynamic range compression, and Inverse Fast Fourier Transform (IFFT).

The estimated power consumption for Hearing Aid Application Specific Integrated Circuits (ASICs) is from 0.5 to 1 mW (@1V power supply). Table 11 presents examples of commercially available microphones.

TABLE 11

| | | Type/ | | |
|---|---|---|---|---|
| Part. no. | Size | Structure | Series | Manufacturer |
| MQM-32325-000 | 3.35 × 2.25 × | omni, MEMS | MQM | Knowles |
| | 0.96 mm | | | |
| P8AC03 | 3.35 × 2.25 × | MEMS | Puma MEMS | Sonion |
| | 0.98 mm | | | |
| P11AC03 | 3.35 × 2.25 × | MEMS | Puma MEMS | Sonion |
| | 1.29 mm | | | |
| O8AC03-MP4 | 3.35 × 2.25 × | Paired MEMS | O series | Sonion |
| | 0.98 mm | | | |
| MMIC271609T4064C0300 | 2.7 × 1.6 × | MEMS | MMIC | TDK InvenSense |
| | 0.89 mm | | | |
| MMIC332509T4070C0300 | 3.35 × 2.25 × | MEMS | MMIC | TDK InvenSense |
| | 0.98 mm | | | |

Condenser microphones are typically the most accurate and smallest currently available (a diaphragm moves and changes a capacitance that generates voltage that will be amplified). Examples of types of condenser microphones include ECM (Electret Condenser Microphone), which is widely used in current technology and characterized by small size, repeatability, performance, and stability over temperature, and MEMS (Micro Electrical Mechanical System), which is driving the revolution in condenser microphones, and allows ultra-small geometries, excellent stability and repeatability, and low power consumption.

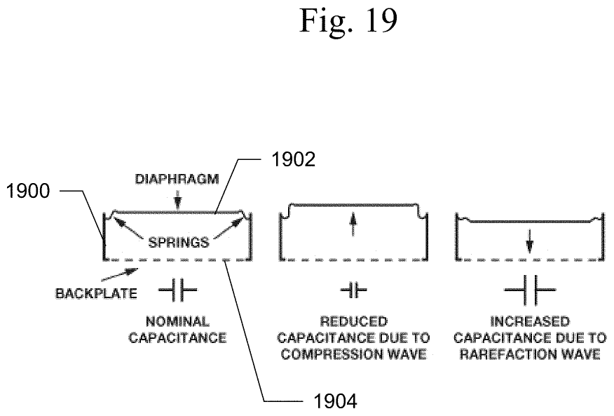

MEMS Technology. MEMS acts as a condenser microphone 1900. A suspended diaphragm 1902 changes a capacity into a cavity (which also has a backplate 1904 acting as an electrode). Air pressure (sounds) changes the distance between the diaphragm and the backplate, which varies the capacitance and thus generates an electrical signal. FIG. 19 illustrates the MEMS microphone functionality. The capacitance of MEMS microphones varies with the pressure level of the acoustic wave. Fabrication wise, MEMS microphones are similar to integrated circuits, and therefore have the advantage of silicon wafer repeatability. MEMs microphones offer features such as ultra small packages, very low power consumption, very low equivalent input noise, improved power supply rejection ratio over ECMs (PSRR typ. −50 dB), low current consumption: 17-20 μA (Zn-air batteries 0.9-1.4V), and good bandwidth, typically 100 Hz-10 KHz. MEMs microphones may support outputs, such as analog (typical output impedance of hundreds ohms), digital Pulse Density Modulation (PDM), digital 12S, etc.

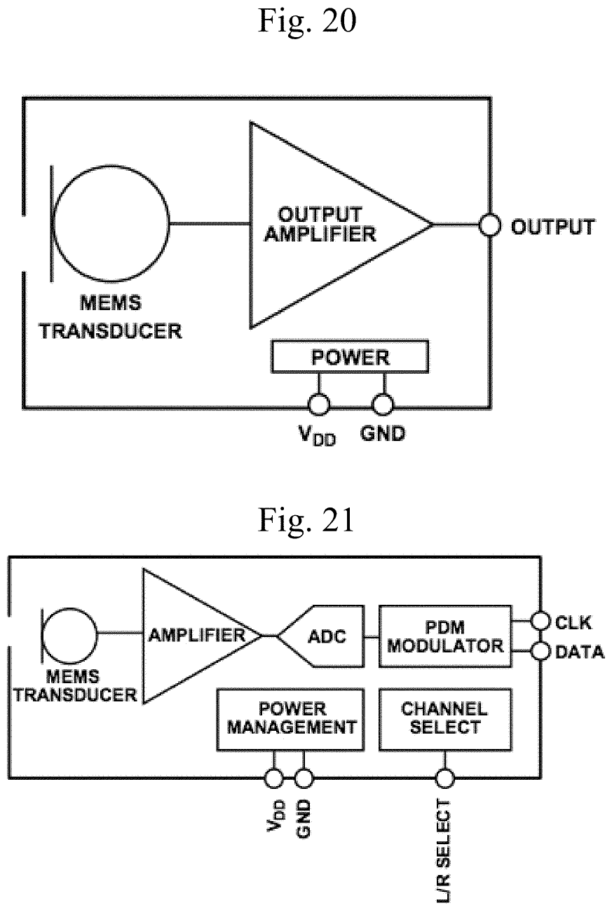

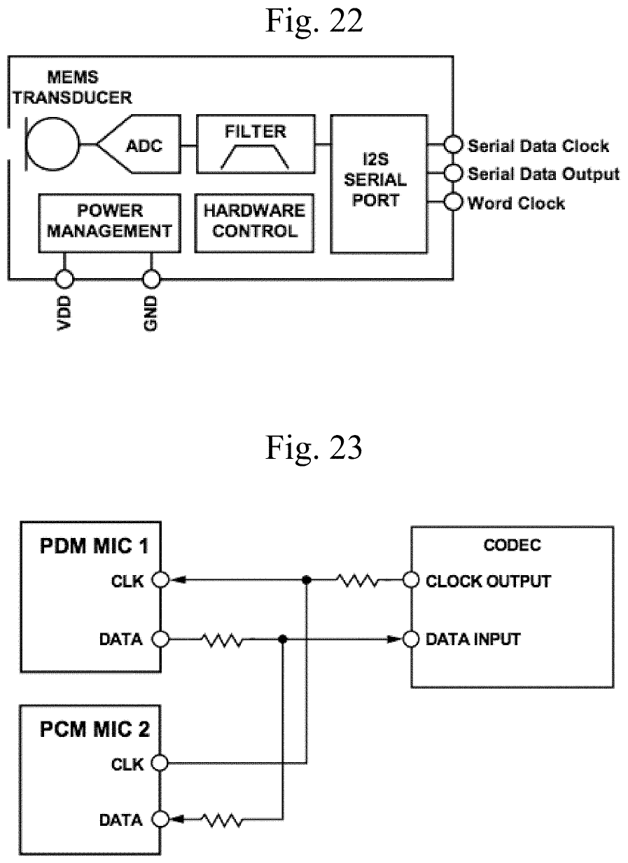

FIG. 20 illustrates an analog MEMS microphone block diagram, FIG. 21 illustrates a digital MEMS microphone with PDM output, and FIG. 22 illustrates a digital MEMS microphone with I2S output.

Typically, care must be taken when choosing MEMS analog or digital microphone technology in hearing aids, in order to avoid interference, such as Electromagnetic Interference, etc., with or from other systems. Filtering and impedance matching is important when designing systems with MEMS and clock and data signals must be properly handled.

PDM is a common digital microphone interface. This format allows two microphones to share a common clock and data line. The topology shown in FIG. 21 maybe used for double system MEMS active filtering microphone technology, as shown in FIG. 23. In this way, directivity with the hearing aid can be achieved, combined with digital PDM-MEMS technology.

Examples of MEMS microphone sizes may include analog MEMS microphone: 3.35×2.5×0.88 mm, digital MEMS microphone: 4×3×1 mm. Using such a microphone, the breath rhythm, for example, may be detected and then measured, with the data being further processed by the SoC.

Speaker. Speaker technologies used in hearing aids are known as SIE (speaker-in-the-ear) for open fit hearing aids or RITE (receiver in the ear hearing aids).

There are also 3 main categories that describe the available technology for hearing aids. Behind-the-Ear (BTE) Hearing Aids are worn with the hearing aid on top of and behind the ear. All of the parts are in the case at the back of the ear and they are joined to the ear canal with a sound tube and a custom mold or tip. In-the-Ear (ITE) Hearing Aids are custom-made devices. All of the electronics sit in a device that fits in the ear. They come in many sizes including Completely in Canal (CIC) and Invisible in Canal (IIC). Receiver-in-Canal (RIC) and Receiver in the Ear (RITE) Hearing Aids are similar in concept to BTE hearing aids, with the exception that the receiver (the speaker) has been removed from the case that sits at the back of the ear. The receiver is fitted in the ear canal or ear and is connected to the case of the hearing aid with a thin wire.

Within these 3 main categories, there are several types of architectures, such as Invisible In Canal (IIC), Completely In Canal (CIC), Mini In Canal (MIC), Microphone In Helix (MIH), In The Ear (ITE), which may be half shell or full shell, Behind The Ear (BTE(, which may be Mini, Standard or Power, Receiver In Canal (RIC), Receiver In The Ear (RITE), etc. Examples of available components are shown in Table 12.

TABLE 12

| Part. no. | Size | Type/structure Series | Manufacturer |
|---|---|---|---|
| BK-21600-000 | 7.87 mm × 5.59 mm × | balanced armature BK | Knowles |
| | 4.04 mm | | |
| FK-23451-000 | 5.00 mm × 2.73 mm × | balanced armature DFK | Knowles |
| | 1.93 mm | | |
| 41A007 | 0.98 × 2.70 × 5.00 | balanced armature 4100 | Sonion |
| | mm | | |
| Molex 504410 | 5.6 × 4.3 × 2.8 mm | balanced armature 504410 | Molex |

Usually the necessary output impedance of the receiver/transducer may be chosen based on the audio driver output characteristics. There is a wide range of output impedances available for such receivers which can be specified for any requirement.

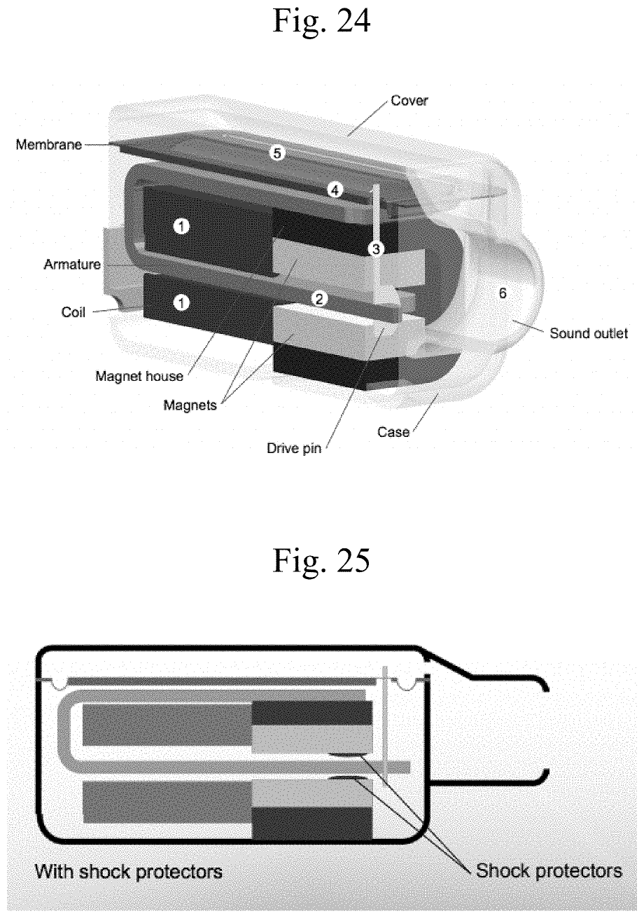

FIGS. 24 and 25 illustrate a typical hearing aid speaker.

Mechanical design. For fitting the electronics inside the earbud, we started the mechanical design based on average dimensions of the ear canal. Air needs to pass past the earbud, and most designs add a tube for this purpose. In embodiments, in order to make the earbud fit to multiple ears is to support it on silicone rubber feet. By using rubber feet, the earbud will fit snugly to many ear shapes, without restricting airflow.

In order for the device to offer EEG and ECG data, embodiments may include several electrodes. Embodiments may utilize metal contact probes. Likewise, embodiments may utilize electrical probes made of electrically conductive silicone rubber, such as the SHIN ETSU® EC-BL, mounted on micro MEMS mechanical transducers.

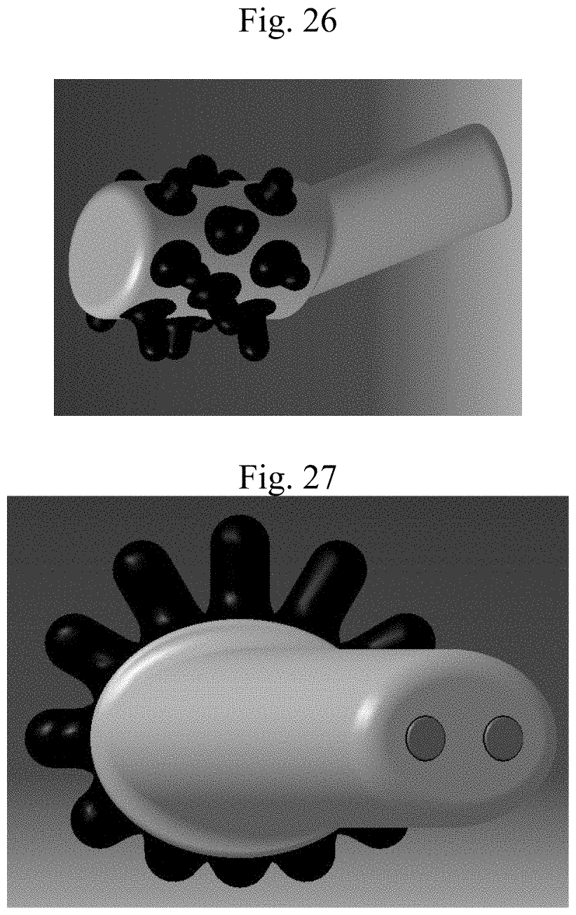

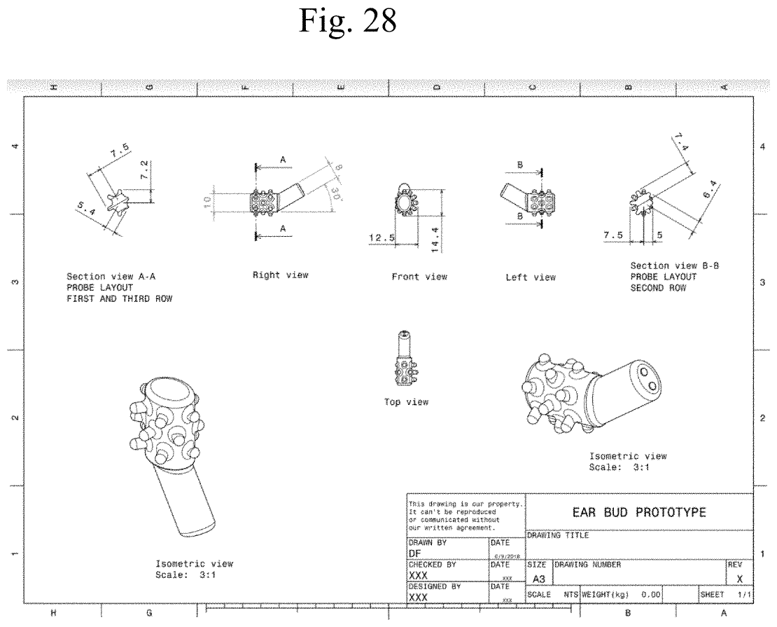

Rubber probes have the advantage of being, at the same time, spacers. Therefore, a device may fit in multiple ear sizes. Further, rubber feet may ensure proper mechanical fixation by pressing against the ear canal. Another advantage of using rubber feet is that air, necessary for good health of the ear, can pass, eliminating a dedicated air tube. FIGS. 26 and 27 illustrate an exemplary embodiment of arrangement for the probes on the device. FIG. 28 illustrates a high-level mechanical drawing of an embodiment.

Electrical Layout. The configuration of the Printed Circuit Board (PCB) layout for embodiments may be based on the dimensions of the parts fitting inside the available space. For example, an approximate available space for an embodiment may be 15 mm (length)×10 mm (height). Table 13 shows exemplary parts for an embodiment of a device.

TABLE 13

| Parts | L × 1 | |
|---|---|---|
| MEMS microphone | 2.7 × 1.6 | mm |
| MMIC271609T | | |
| Speaker | 5 × 2.7 | mm |
| 41A007 (Sonion) | | |
| Temp, sensor | | |
| TMP006 (T.I.) -------- | 1.56 × 1.56 | mm |
| ----> | | |
| MLX90632 (Melexis) ------- | 3 × 3 | mm |
| -----> | | |
| Bluetooth S.O.C (System On Chip) | 2.35 × 2.32 | mm |
| RSL10 (ON) | | |
| Operational Amplifier | | |
| ADA4505-4 (ADI) | BGA 3 × 1.5 × 0.65(1 pcs.) | |
| ADA4505-1 (ADI) | BGA 1.45 × 0.95 × 0.65(1 pcs.) | |
| Accelerometer | 1.085 × 1.085 | mm |
| MC3571 (MCube) | | |
| Photodiode and Single-Supply | 3.3 × 5.6 × 1.3 | mm |
| Transimpedance Amplifier - | | |
| PPG & EEG | | |
| MAX86150 - OLGA/14 | | |

Power Management. An exemplary power consumption profile may be estimated given some common-sense duty cycles, as shown in Table 14. For example, a duty cycle of 0.01 for the temperature sensor TMP006 in 24 hours would correspond to 14.4 minutes.

TABLE 14

| | | | | | Power | t max |
|---|---|---|---|---|---|---|
| | | Duty | Current | Voltage | [mW] | Vbat |
| Device | No | cycle | [μA] | [V] | Vba | min |
| MMIC271609T | 2 | 1 | 90 | 3.3 | 0.594 | 4.15 |
| | | | | | | 3.6 |
| TMP006 | 1 | 0.01 | 90 | 3.3 | 0.00297 | 4.15 |
| | | | | | | 3.6 |
| RSUORX | 1 | 0.014 | 3000 | 3.3 | 0.1386 | 4.15 |
| | | | | | | 3.6 |
| RSUOTX | 1 | 0.014 | 4600 | 3.3 | 0.21252 | 4.15 |
| | | | | | | 3.6 |
| RSL1 OuP | 1 | 0.02 | 1800 | 3.3 | 0.1188 | 4.15 |
| | | | | | | 3.6 |
| RSUOspk drv | 1 | 0.1 | 6200 | 3.3 | 2.046 | 4.15 |
| | | | | | | 3.6 |
| ADA4505 | 5 | 1 | 10 | 3.3 | 0.165 | 4.15 |
| | | | | | | 3.6 |
| MC3571 | 1 | 0.1 | 36 | 3.3 | 0.01188 | 4.15 |
| | | | | | | 3.6 |
| MAX86150 | 1 | 0.014 | 750 | 3.3 | 0.03465 | 4.15 |
| LEO | | | | | | 3.6 |
| MAX86150 | 1 | 0.014 | 750 | 1.8 | 0.0189 | 4.15 |
| ECG | | | | | | 3.6 |

Embodiments may utilize various use-cases and daily running time based on goals that may be validated, for example, by a medical professional, for each sensor. These use-cases and running times may influence the power consumption profile. For example, the power budget shown in Table 14 may provide 43 hours of continuous running.

Embedded System Considerations. Embodiments may utilize a system on chip with low power consumption, audio processing, and Bluetooth 5 compatibility. Embodiments may utilize the RSL10 from ON Semiconductor.

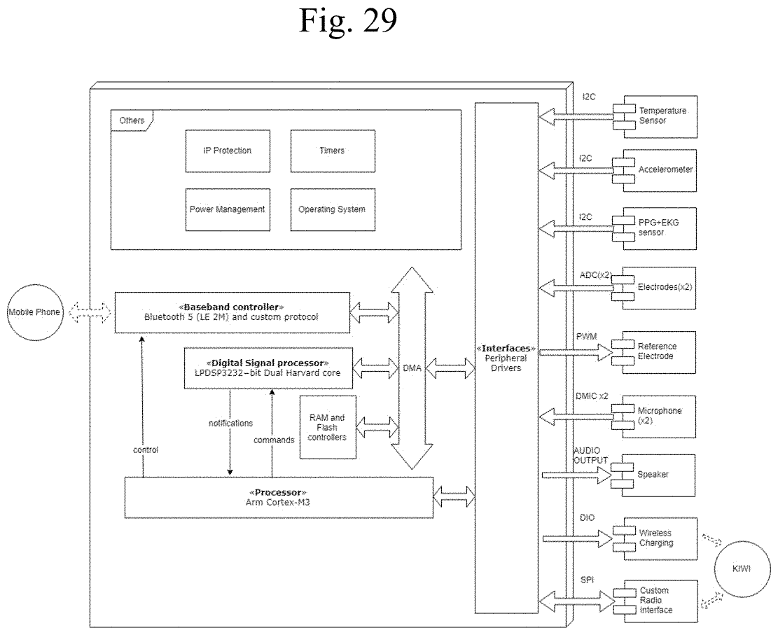

FIG. 29 shows an overview of the exemplary embodiment of an embedded system architecture, with examples of components that may be used.

Sensorics. In embodiments, data captured by the multiple sensors may be read and stored by the embedded system. For example, embodiments may include a temperature sensor—data read via I²C, a 2-wire protocol, an accelerometer—data read via I²C, a 2-wire protocol, a dedicated PPG+ECG sensor—data read via I²C, a 2-wire protocol, electrodes—voltage read via ADC (note: the reference electrode can be manipulated via pulse-width modulation). For example, the RSL10 features all the micro peripherals described above. Software drivers are also available from ON Semiconductor.

Audio. Embodiments may utilize a specialized digital signal processor (DSP) for an application in which handles audio signals. Having such dedicated hardware integrated into the embedded system may provide several advantages, such as economy of processing power—the main processor is freed up for other tasks, no need for hardware filters—fewer physical components which leads to a simpler design, greater miniaturization Signal filtering and real time noise cancellation.

For example, the RSL10 includes such a digital signal processor—the LPDSP32. It is a is a low power, programmable, pipelined DSP that uses a dual-Harvard, dual-MAC architecture to efficiently process 32-bit signal data. This processor supports multiple audio codecs (available to customers through libraries that are included in RSL10's development tools) and can be programmed independently through a separate JTAG connection.

In embodiments, data from the two omnidirectional microphones may be read via standard DMIC (digital microphone inputs) interface. This includes an input pin for data and an output pin for clock. The RSL10 (and other microcontrollers specialized for audio applications) provide a dedicated DMIC block whose signals can be routed to standard DIO pins.

For sound output via a speaker, a standard output driver is required. The output driver provides a mono digital audio output. This output driver can be connected to drive one or more DIO pairs, which are used as the driver for a speaker or receiver. The RSL10 comes with a dedicated output driver.

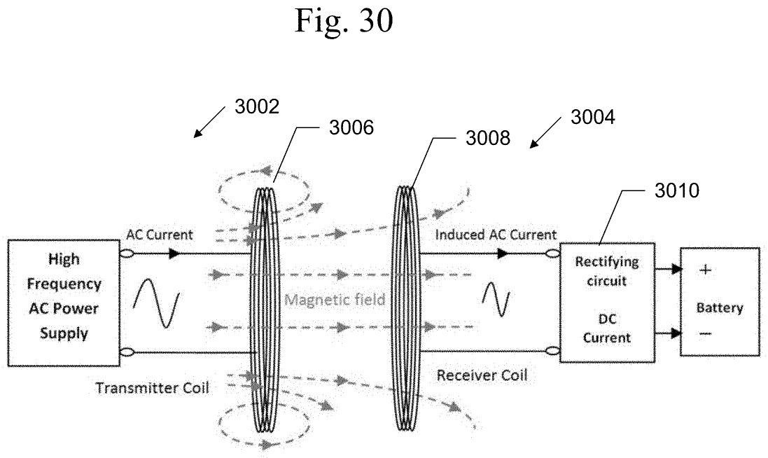

Wireless Charging. An exemplary block diagram of a wireless charging system is shown in FIG. 30. Embodiments may include two individual power blocks, one for power transmission 3002 and one for power reception 3004. The transmitting coil 3006 may generate a magnetic field and thus induce AC current into the receiver coil 3008. The flux density of the transmitting coil 3006 decreases with the geometrical displacement, angle, and distance from receiver coil 3008. Due to the variable magnetic flux, receiver coil 3008 may generate an induced voltage at its terminals. The output voltage at the receiver coil may rectified, boosted, or regulated by a dedicated battery charger circuit 3010.

Other features. Embodiments may provide additional features, such as:

Power Management—desirable for any low-power application; all micro's provide the possibility to reduce power consumption via different run modes and disabling peripherals; a software strategy to take advantage of these features can be implemented.

Security—data transmitted over Bluetooth may be encoded via different methods for security purposes.

Data integrity—mechanisms to ensure integrity of the large amount of sensor data may be implemented (example CRC).

Operating system & Timers—may be used for accurate timing of processing tasks; solutions for operating systems are either provided by the manufacturer or commercially available

Flash Storage—important data may be stored to non-volatile memory, making it available over multiple use-cycles (example: a user-specific “baseline” for blood pressure could be measured and programmed to the device; this would allow the device itself to calculate deviations and issue specific warnings if measured values exceed a certain tolerance; this example could apply to all sensoric data)

Flashing Protocol—a custom SW communication protocol may be implemented (over Bluetooth) which would allow over-the-air updates

Embodiments may also provide additional features such as: immersive selective music to provide an audio experience with custom fit earbuds, which may include an integrated equalizer to customize the sound; connected voice control to provide voice control over surroundings and to interact with the Web and the smart objects in the vicinity; seamless real-time translation which may provide the capability to understand any language by listening to what other people are saying, in any selected language, in real-time; disturbance-free communication which may capture voice through the inner-ear providing the ability to speak softly, rather than shout; augmented digital hearing may provide the capability to adjust the volume of natural hearing to a desired level, reducing the stress and distraction of noisy environments; dynamic hearing protection may adapt to specified noise-level requirements by setting an accepted level of sound in dB and automatically maintaining the selected level; dynamic environment awareness may provide the capability to dynamically blend desired audio quality and outside sounds; hearing protection may provide the capability to attenuate outside noise while providing desired audio.

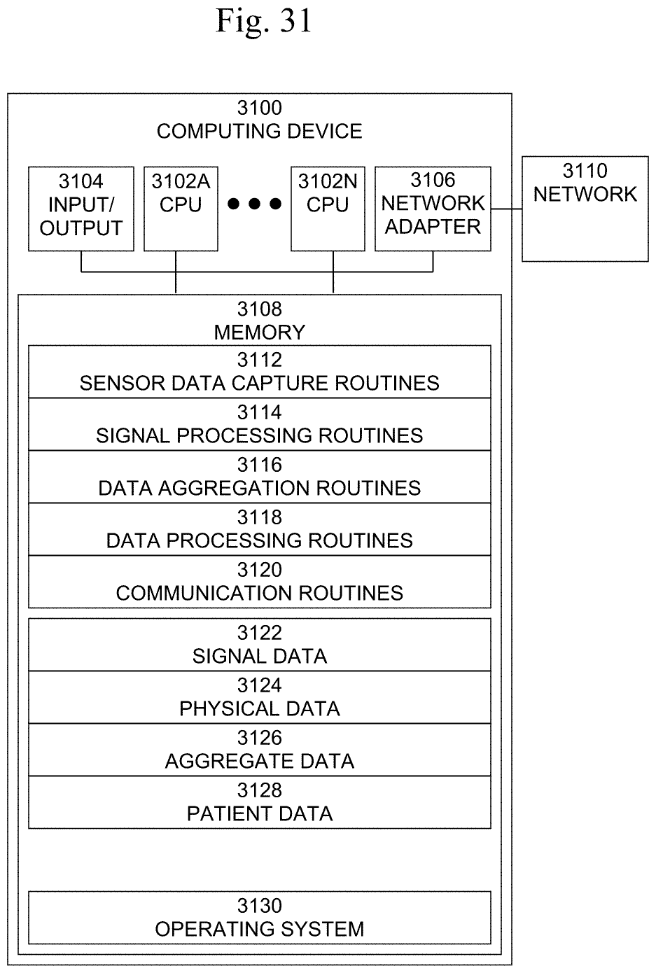

An exemplary block diagram of a computer system 3100, in which processes involved in the embodiments described herein may be implemented, is shown in FIG. 31. Computer system 3100 is typically a programmed general-purpose computer system, such as an embedded processor, system on a chip, personal computer, workstation, server system, and minicomputer or mainframe computer. Computer system 3100 may include one or more processors (CPUs) 3102A-3102N, input/output circuitry 3104, network adapter 3106, and memory 3108. CPUs 3102A-3102N execute program instructions in order to carry out the functions of the present invention. Typically, CPUs 3102A-3102N are one or more microprocessors, microcontrollers, processor in a System-on-chip, etc. FIG. 31 illustrates an embodiment in which computer system 3100 is implemented as a single multi-processor computer system, in which multiple processors 3102A-3102N share system resources, such as memory 3108, input/output circuitry 3104, and network adapter 3106. However, the present invention also contemplates embodiments in which computer system 3100 is implemented as a plurality of networked computer systems, which may be single-processor computer systems, multi-processor computer systems, or a mix thereof.

Input/output circuitry 3104 provides the capability to input data to, or output data from, computer system 3100. For example, input/output circuitry may include input devices, such as sensors, microphones, keyboards, mice, touchpads, trackballs, scanners, etc., output devices, such as speakers, video adapters, monitors, printers, etc., and input/output devices, such as, modems, etc. Network adapter 3106 interfaces device 3100 with a network 3110. Network 3110 may be any public or proprietary LAN or WAN, including, but not limited to the Internet.

Memory 3108 stores program instructions that are executed by, and data that are used and processed by, CPU 3102 to perform the functions of computer system 3100. Memory 3108 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 3108 may vary depending upon the function that computer system 3100 is programmed to perform. One of skill in the art would recognize that routines, along with the memory contents related to those routines, may not typically be included on one system or device, but rather are typically distributed among a plurality of systems or devices, based on well-known engineering considerations. The present invention contemplates any and all such arrangements.

In the example shown in FIG. 31, memory 3108 may include sensor data capture routines 3112, signal processing routines 3114, data aggregation routines 3116, data processing routines 3118, signal data 3122, physical data 3124, aggregate data 3126, patient data 3128, and operating system 3130. For example, sensor data capture routines 3112 may include routines to receive and process signals from sensors, such as those described above, to form signal data 3122. Signal processing routines 3114 may include routines to process signal data 3120, as described above, to form physical data 3124. Data aggregation routines 3116 may include routines to process physical data 3124, as described above, to generate aggregate data 3126. Data processing routines 3118 may include routines to process physical data 3124, aggregate data 3126, and/or patient data 3128. Operating system 3120 provides overall system functionality.

As shown in FIG. 31, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A system for monitoring human body activity comprising:
a device adapted to be mounted in an ear of a human, the device comprising a plurality of sensors, each sensor adapted to monitor a physical or physiological parameter of the human and output a signal representing the monitored physical or physiological parameter;
a data collection device adapted to receive the plurality of signals from the plurality of sensors and to process the signals to form digital data representing the monitored physical or physiological parameters; and
a data processing device adapted to process digital data representing the monitored physical or physiological parameters to determine a condition or activity of the human body.

2. The system of claim 1, wherein the device adapted to be mounted in an ear of a human further comprises a first portion adapted to be inserted in an ear canal of the human and a second portion adapted to protrude from the ear of the human; and
the first portion comprises a plurality of protrusions, wherein at least some of the plurality of protrusions comprise at least one sensor.

3. The system of claim 2, wherein the sensors comprise at least a plurality of sensors selected from a group comprising: audio sensors, video sensors, EEG sensors, ECG sensors, heart rate sensors, breathing rate sensors, blood pressure sensors, body temperature sensors, head movement sensors, body posture sensors, and blood oxygenation levels sensors.

4. The system of claim 2, wherein each protrusion comprises an electrically conductive rubber portion and an electrically isolated shell, wherein the electrically conductive rubber portion is adapted to be a dry electrode and to sense signals to be used for at least one of electroencephalography and electrocardiography.

5. The system of claim 4, wherein each protrusion further comprises microelectromechanical systems transducer comprising a mechanical transducer adapted to output an electrical signal representing a mechanical signal and an electrode adapted to output electrical signals received from a skin surface of the human body.

6. The system of claim 5, wherein the electrode is a flexible electrode and the microelectromechanical systems transducer is further adapted to output an electrical signal representative of physical movement of the flexible electrode.

7. The system of claim 6, wherein the data processing device is further adapted to determine artefacts of the physical movement that may be present in the electrical signal output from the flexible electrode, and to subtract the artefacts from the electrical signal output from the flexible electrode, to form a cleaner signal.

8. The system of claim 2, further adapted to perform at least some of blood pressure measurement using Pulse Transit Time (PTT) and/or Pulse Wave Velocity (PWV), tympanic membrane infrared temperature measurement, accelerometer measuring of heart rate (HR), breathing rate (BR) and activity tracking, Photoplethysmography (PPG) optical measurement of blood volume changes, hearing aid functions, and music streaming capabilities with noise cancellation.

9. The system of claim 2, wherein the second portion comprises a battery.

10. The system of claim 2, wherein the device adapted to be mounted in an ear of a human is further adapted to provide at least one of: an audio experience using custom fit earbuds, customized sound using an integrated equalizer, connected voice control, real-time translation, disturbance-free communication using inner-ear voice capture; augmented digital hearing to adjust the volume of natural hearing to a desired level, dynamic hearing protection by setting an accepted level of sound in dB and automatically maintaining the selected level, dynamic environment awareness by dynamically blending desired audio quality and outside sounds, and hearing protection by attenuating outside noise while providing desired audio.

11. A computer-implemented method for monitoring human body activity comprising:
receiving from each of a plurality of sensors a signal representing a monitored physical or physiological parameter, wherein each sensor is adapted to monitor a physical or physiological parameter of the human and output a signal representing the monitored physical or physiological parameter;
processing the received signals to form digital data representing the monitored physical or physiological parameters; and
processing digital data representing the monitored physical or physiological parameters to determine a condition or activity of the human body.

12. The method of claim 11, wherein each sensor is included in a protrusion included in a first portion of a device adapted to be mounted in an ear of a human, the device comprising a first portion adapted to be inserted in an ear canal of the human and a second portion adapted to protrude from the ear of the human.

13. The method of claim 12, wherein the sensors comprise at least a plurality of sensors selected from a group comprising: audio sensors, video sensors, EEG sensors, ECG sensors, heart rate sensors, breathing rate sensors, blood pressure sensors, body temperature sensors, head movement sensors, body posture sensors, and blood oxygenation levels sensors.

14. The method of claim 12, wherein each protrusion comprises an electrically conductive rubber portion and an electrically isolated shell, wherein the electrically conductive rubber portion is adapted to be a dry electrode and to sense signals to be used for at least one of electroencephalography and electrocardiography.

15. The method of claim 14, wherein each protrusion further comprises microelectromechanical systems transducer comprising a mechanical transducer adapted to output an electrical signal representing a mechanical signal and an electrode adapted to output electrical signals received from a skin surface of the human body.

16. The method of claim 15, wherein the electrode is a flexible electrode and the microelectromechanical systems transducer is further adapted to output an electrical signal representative of physical movement of the flexible electrode.

17. The method of claim 16, wherein the data processing device is further adapted to determine artefacts of the physical movement that may be present in the electrical signal output from the flexible electrode, and to subtract the artefacts from the electrical signal output from the flexible electrode, to form a cleaner signal.

18. The method of claim 12, wherein the device adapted to be mounted in an ear of a human is further adapted to perform at least some of blood pressure measurement using Pulse Transit Time (PTT) and/or Pulse Wave Velocity (PWV), tympanic membrane infrared temperature measurement, accelerometer measuring of heart rate (HR), breathing rate (BR) and activity tracking, Photoplethysmography (PPG) optical measurement of blood volume changes, hearing aid functions, and music streaming capabilities with noise cancellation.

19. The method of claim 12, the device adapted to be mounted in an ear of a human is further adapted to provide at least one of: an audio experience using custom fit earbuds, customized sound using an integrated equalizer, connected voice control, real-time translation, disturbance-free communication using inner-ear voice capture; augmented digital hearing to adjust the volume of natural hearing to a desired level, dynamic hearing protection by setting an accepted level of sound in dB and automatically maintaining the selected level, dynamic environment awareness by dynamically blending desired audio quality and outside sounds, and hearing protection by attenuating outside noise while providing desired audio.

20. The method of claim 12, wherein the second portion comprises a battery.
